(19) United States
(12) Patent Application Publication
(10) Pub. No.: US 2025/0090563 A1
(43) Pub. Date: Mar. 20, 2025

(54) COMPOSITION FOR PREVENTION, AMELIORATION OR TREATMENT OF INFLAMMATORY BOWEL DISEASE COMPRISING GALACTO-OLIGOSACCHARIDE

(71) Applicant: NEO CREMAR CO., LTD., Seoul (KR)

(72) Inventors: Jae-Hwan KIM, Seoul (KR); Kwang-Soon SHIN, Seoul (KR); Jung-Cheul SHIN, Seoul (KR); Hye-Ryung PARK, Seoul (KR); Na-Ri KIM, Seoul (KR); Young-Jin BAEK, Seoul (KR); Dong-Hyun EOM, Gyeonggi-do (KR)

(21) Appl. No.: 18/726,900

(22) PCT Filed: Jan. 21, 2022
(86) PCT No.: PCT/KR2022/001168

§ 371 Date: Jul. 5, 2024

(30) Foreign Application Priority Data
Jan. 7, 2022 (KR) 10-2022-0002842

(51) Int. Cl. A61K 31/702; A61P 1/00; A61P 29/00

(52) U.S. Cl. CPC A61K 31/702; A61P 1/00; A61P 29/00

(57) ABSTRACT

The present disclosure relates to a composition for the prevention, amelioration or treatment of inflammatory bowel disease, comprising a galacto-oligosaccharide as an active ingredient. The galacto-oligosaccharide composition of the present disclosure has an anti-inflammatory effect of ameliorating DAI and reducing the levels of inflammatory cytokines and MPO in inflammatory bowel disease model mice, and thus can be effectively used as a drug or food for the prevention, amelioration or treatment of inflammatory bowel disease.

This patent application claims priority to and the benefit of Korean Patent Application No. 10-2022-0002842, filed with the Korean Intellectual Property Office on Jan. 7, 2022, the disclosure of which is incorporated herein by reference.

### TECHNICAL FIELD

The present disclosure relates to a composition comprising galacto-oligosaccharide for the prevention, amelioration, or treatment of inflammatory bowel disease.

### BACKGROUND ART

Recently, with the rapid Westernization of dietary habits, the incidence of colon diseases in Korea has sharply increased. In particular, colorectal cancer and inflammatory bowel disease (IBD) have increased at an alarming rate. Western dietary patterns change the composition of the gut microbiota, leading to a significant deterioration of the gut environment due to metabolic changes. These changes have drawn significant attention due to their association with inflammatory metabolic diseases, such as obesity, and other illnesses (Nature, 2012).

Inflammatory bowel disease (IBD) affects about 50,000 patients in Korea, with 20-40-year-old patients accounting for over 50% of all cases. IBD patients can generally be classified into two groups: those with ulcerative colitis and those with Crohn's disease. While these diseases vary in terms of lesion location and progression, ulcerative colitis is more prevalent in Koreans. Ulcerative colitis, which is frequently diagnosed in Korea, results in the loss of the intestinal mucosa layer, increased inflammation, and disruption of the tight junctions between intestinal epithelial cells, leading to leaky gut syndrome. This condition allows the entry of lipopolysaccharides (LPS) and toxins into the gut, causing continuous inflammation and allergic reactions that can affect other organs, including the immune system, brain, kidneys, and skeletal muscles. Therefore, to address these intestinal diseases, a fundamental approach is required beyond traditional chemical synthetic agents (anti-inflammatory drugs) or biological agents (TNF-targeted molecular agents).

### RELATED ART DOCUMENT

### Non-Patent Literature

Zhao et al., Nature Reviews Microbiology, 2013

### DISCLOSURE OF INVENTION

### Technical Problem

Leading to the present disclosure, thorough and intensive research conducted by the present inventors with the aim to develop an effective treatment for inflammatory bowel disease by enhancing the intestinal immune function or improving the intestinal environment through the production of short-chain fatty acids (SCFA) resulted in the finding that the high-purity oligosaccharide designed by the present inventors is effective in treating inflammatory bowel disease and positively influences the growth of beneficial intestinal bacteria and SCFA formation.

Therefore, the present disclosure is to provide a pharmaceutical composition comprising galacto-oligosaccharide as an active ingredient for the prevention or treatment of inflammatory bowel disease.

Also, the present disclosure is to provide a food composition comprising galacto-oligosaccharide as an active ingredient for the prevention or amelioration of inflammatory bowel disease.

### Solution to Problem

According to one aspect thereof, the present disclosure provides a pharmaceutical composition comprising galacto-oligosaccharide is provided as an active ingredient for the prevention or treatment of inflammatory bowel disease.

As used herein, the term “galacto-oligosaccharide” (GOS) does not refer solely to pure galacto-oligosaccharide, so it is referred to as galacto-oligosaccharide or galacto-oligosaccharide composition.

In an embodiment of the present disclosure, the galacto-oligosaccharide composition has a purity of 70% or higher. The purity of the galacto-oligosaccharide composition refers to the weight percentage of i) monosaccharides such as glucose and galactose and ii) oligosaccharides including a) disaccharides except for lactose used as a raw material for galacto-oligosaccharide production, b) trisaccharides, and c) tetra- or higher saccharides, based on the total weight of the galacto-oligosaccharide composition.

In an embodiment of the present disclosure, the galacto-oligosaccharide composition includes monosaccharides, disaccharides, trisaccharides, and higher oligosaccharides.

The monosaccharides are glucose and galactose. The disaccharides include lactose, sucrose, and maltose. The trisaccharides include galactosyllactose, nigerotriose, maltotriose, melezitose, maltotriulose, raffinose, kestose, but are not limited thereto. The tetrasaccharides include acarbose, stachyose, lychnose, maltotetraose, nigerotetraose, nystose, and sesamose, but are not limited thereto.

In a specific embodiment of the present disclosure, the purity of the galacto-oligosaccharide composition is 70% to 95%, 70% to 90%, 70% to 85%, 70% to 80%, and 70% to 75%, and more specifically, 70% to 95%, 71% to 95%, 72% to 95%, 73% to 95%, 74% to 95%, 75% to 95%, 76% to 95%, 77% to 95%, 78% to 95%, 79% to 95%, 80% to 95%, 85% to 95%, 90% to 95%, 70% to 90%, 71% to 90%, 72% to 90%, 73% to 90%, 74% to 90%, 75% to 90%, 76% to 90%, 77% to 90%, 78% to 90%, 79% to 90%, 80% to 90%, 81% to 90%, 82% to 90%, 83% to 90%, 84% to 90%, 85% to 90%, 70% to 85%, 71% to 85%, 72% to 85%, 73% to 85%, 74% to 85%, 75% to 85%, 76% to 85%, 77% to 85%, 78% to 85%, 79% to 85%, 80% to 85%, 81% to 85%, 82% to 85%, 83% to 85%, 84% to 85%, 70% to 84%, 71% to 84%, 72% to 84%, 73% to 84%, 74% to 84%, 75% to 84%, 76% to 84%, 77% to 84%, 78% to 84%, 79% to 84%, 80% to 84%, 81% to 84%, 82% to 84%, 83% to 84%, 70% to 82%, 71% to 82%, 72% to 82%, 73% to 82%, 74% to 82%, 75% to 82%, 76% to 82%, 77% to 82%, 78% to 82%, 79% to 82%, 80% to 82%, 81% to 82%, 70% to 80%, 71% to 80%, 72% to 80%, 73% to 80%, 74% to 80%, 75% to 80%, 76% to 80%, 77% to 80%, 78% to 80%, 79% to 80%, 70%, 71%, 72%, 73%, 74%, 75%, 76%, 77%, 78%, 79%, 80%, 81%, 82%, 83%, 84%, or 85%, but with no limitations thereto.

In an embodiment of the present disclosure, galactosyllactose is a trisaccharide composed of lactose and galactose. Galactosyllactose, which is a component contained in large amounts in a mother's breast milk immediately after birth, is known to have various physiological activities.

In a specific embodiment of the present disclosure, the galactosyllactose is 4′-galactosyllactose, but with no limitations thereto.

The galacto-oligosaccharide composition of the present disclosure includes 4′-galactosyllactose in an amount of 12% by weight, based on the total weight thereof. In another embodiment of the present disclosure, the 4′-galactosyllactose is contained in an amount of 12%, 13%, 14%, 15%, 16%, 17%, 18%, 19%, 20%, 21%, 22%, 23%, 24%, 25%, or higher, but with no limitations thereto.

With a very high content of the 4′-galactosyllactose component, the galacto-oligosaccharide composition of the present disclosure exhibits superb prophylactic, palliative, and therapeutic effects on inflammatory bowel disease even when being similar in purity to pre-existing commercially available galacto-oligosaccharide compositions.

More specifically, the galacto-oligosaccharide composition may contain 4′-galactosyllactose in an amount of 12% to 20%, 13% to 20%, 14% to 20%, 15% to 20%, 16% to 20%, 17% to 20%, 18% to 20%, 19% to 20%, 12% to 22%, 12% to 22%, 13% to 22%, 14% to 22%, 15% to 22%, 16% to 22%, 17% to 22%, 18% to 22%, 19% to 22%, 12% to 23%, 12% to 23%, 13% to 23%, 14% to 23%, 15% to 23%, 16% to 23%, 17% to 23%, 18% to 23%, 19% to 23%, 20% to 23%, 12% to 24%, 12% to 24%, 13% to 24%, 14% to 24%, 15% to 24%, 16% to 24%, 17% to 24%, 18% to 24%, 19% to 24%, 20% to 24%, 21% to 24%, 12% to 25%, 13% to 25%, 14% to 25%, 15% to 25%, 16% to 25%, 17% to 25%, 18% to 25%, 19% to 25%, 20% to 25%, 21% to 25%, or 22% to 25%, based on the total weight thereof, but with no limitations thereto.

In an embodiment of the present disclosure, the galacto-oligosaccharide composition may contain monosaccharides in an amount of 5% or less, for example, 0 to 5%, 0 to 4.5%, 0 to 4%, 0 to 3.5%, 0 to 3%, 0 to 2.5%, 0 to 2%, 0 to 1%, 0.1 to 5%, 0.1 to 4.5%, 0.1 to 4%, 0.1 to 3.5%, 0.1 to 3%, 0.1 to 2.5%, 0.1 to 2%, 0.1 to 1%, 1 to 5%, 1 to 4.5%, 1 to 4%, 1 to 3.5%, 1 to 3%, 1 to 2.5%, 1 to 2%, 2 to 5%, 2 to 4.5%, 2 to 4%, 2 to 3.5%, 2 to 3%, 3 to 5%, 3 to 4.5%, or 3 to 4%, based on the total weight thereof, but with no limitations thereto.

In an embodiment of the present disclosure, the galacto-oligosaccharide composition may contain disaccharides except for lactose in an amount of 10 to 40%, 15 to 40%, 20 to 40%, 25 to 40%, 30 to 40%, 10 to 35%, 15 to 35%, 20 to 35%, 25 to 35%, 30 to 35%, 10 to 30%, 15 to 30%, 16 to 30%, 17 to 30%, 18 to 30%, 19 to 30%, 20 to 30%, 21 to 30%, 22 to 30%, 23 to 30%, 24 to 30%, 25 to 30%, 10 to 25%, 15 to 25%, 16 to 25%, 17 to 25%, 18 to 25%, 19 to 25%, 20 to 25%, 21 to 25%, 22 to 25%, 23 to 25%, or 24 to 25%, based on the total weight thereof, but with no limitations thereto.

In an embodiment of the present disclosure, the galacto-oligosaccharide composition may contain trisaccharides in an amount of 20 to 40%, 25 to 40%, 30 to 40%, 10 to 35%, 15 to 35%, 16 to 35%, 17 to 35%, 18 to 35%, 19 to 35%, 20 to 35%, 21 to 35%, 22 to 35%, 23 to 35%, 24 to 35%, 25 to 35%, 26 to 35%, 27 to 35%, 28 to 35%, 29 to 35%, 30 to 35%, 31 to 35%, 32 to 35%, 33 to 35%, 34 to 35%, 10 to 30%, 15 to 30%, 16 to 30%, 17 to 30%, 18 to 30%, 19 to 30%, 20 to 30%, 21 to 30%, 22 to 30%, 23 to 30%, 24 to 30%, or 25 to 30%, based on the total weight thereof, but with no limitations thereto.

In an embodiment of the present disclosure, the galacto-oligosaccharide composition may contain tetra- or higher saccharides in an amount of 10 to 40%, 15 to 40%, 20 to 40%, 25 to 40%, 30 to 40%, 10 to 35%, 15 to 35%, 20 to 35%, 25 to 35%, 30 to 35%, 10 to 30%, 15 to 30%, 16 to 30%, 17 to 30%, 18 to 30%, 19 to 30%, 20 to 30%, 21 to 30%, 22 to 30%, 23 to 30%, 24 to 30%, 25 to 30%, 10 to 25%, 15 to 25%, 16 to 25%, 17 to 25%, 18 to 25%, 19 to 25%, 20 to 25%, 21 to 25%, 22 to 25%, 23 to 25%, or 24 to 25%, based on the total weight thereof, but with no limitations thereto.

As used herein, the term “inflammatory bowel disease” refers to conditions that cause inflammation of the intestines, both small and large, and includes diseases characterized by abnormal, chronic inflammation within the intestines that alternates between remission and relapse. The term encompasses specific enteritis of known causes, nonspecific enteritis of unknown causes, and enteritis arising from other diseases, such as intestinal Behcet's disease.

In an embodiment of the present disclosure, the inflammatory bowel disease is selected from the group consisting of ulcerative colitis, Crohn's disease, intestinal Behcet's disease, indeterminate colitis, bacterial enteritis, viral enteritis, amoebic colitis, hemorrhagic rectal ulcers, leaky gut syndrome, ischemic colitis, and tuberculous enteritis, but are not limited thereto. More specifically, the inflammatory bowel disease is ulcerative colitis or Crohn's disease.

In one embodiment of the present disclosure, the galacto-oligosaccharide composition inhibits (myeloperoxidase (MPO) activity and suppresses neutrophil infiltration in inflamed tissues.

In another embodiment of the present disclosure, the galacto-oligosaccharide composition reduces the levels of inflammation-inducing cytokines (TNF-alpha and IL-6).

In another embodiment of the present disclosure, the galacto-oligosaccharide composition effectively lowers disease activity index (DAI) caused by inflammatory bowel disease.

In another embodiment of the present disclosure, the galacto-oligosaccharide composition exhibits the effect of reversing the colon length reduction attributed to inflammatory bowel disease.

In another embodiment of the present disclosure, the galacto-oligosaccharide composition promotes (or increases) the production of short-chain fatty acids in the intestines of patients with inflammatory bowel disease.

Specifically, the galacto-oligosaccharide composition of the present disclosure increases the production of butyric acid.

The criteria for determining the increase or decrease of the above indicators (such as inflammatory cytokine levels, disease activity index, colon length, and short-chain fatty acid levels) following administration of the galacto-oligosaccharide composition are the indicator values of the normal group or the disease-induced negative control group.

Fatty acids determine the structure and nature of lipids, and nutritional characteristics differ depending on the type of fatty acid. Based on the carbon chain length, fatty acids are classified as short-chain if they contain fewer than eight carbon atoms, medium-chain if they have 8-14, and long-chain if they have 16 or more. Fatty acids are further categorized as monounsaturated (MUFA) or polyunsaturated (PUFA) based on the number of double bonds. Polyunsaturated fatty acids are divided into ω3 and ω6, depending on where the first double bond occurs from the CH₃ terminal. Monounsaturated fatty acids are differentiated into cis and trans isomers based on the nature of their double bonds.

The functionality of short-chain fatty acids (SCFAs) has been documented in many studies. Besides serving as an energy source for colon epithelial cells, SCFAs stimulate peristalsis to aid digestion and absorption, acidify the intestines to inhibit harmful bacteria, and protect against infections, thus playing an important role.

It is well known that foods high in fiber are beneficial for preventing obesity and metabolic disorders. Fiber can be broadly divided into insoluble dietary fibers like cellulose and lignin, and soluble dietary fibers like pectin, hemicellulose, galacto-oligosaccharides, and fructo-oligosaccharides. Soluble dietary fiber, which includes the galacto-oligosaccharides of the present disclosure, is fermented by gut microbes to produce short-chain fatty acids (SCFAs) such as acetic, propionic, and butyric acids. SCFAs regulate histone acetylation to induce epigenetic changes, and bind to G-protein-coupled receptors (GPCRs), FFAR3 and FFAR2. By influencing these receptors, SCFAs regulate glucagon-like peptide 1 (GLP-1) secretion, fat synthesis, and gut transit time, thus controlling systemic energy homeostasis.

Additionally, SCFAs are absorbed in the colon and play a vital role in inhibiting cholesterol biosynthesis in the liver and reducing the absorption of cholesterol by the body. Recent findings show that SCFAs activate G-protein receptor 41 (GPR41) in the sympathetic nervous system. In conditions of excess energy, GPR41 stimulates sympathetic nerves to increase energy expenditure. In starvation states, ketone bodies produced by the liver suppress sympathetic nerves through GPR41, reducing energy expenditure. Therefore, SCFAs are crucial for energy homeostasis, gaining significant attention as important factors in gut health.

In an embodiment of the present disclosure, the galacto-oligosaccharide composition normalizes the gut microbiome distribution of mice with inflammatory bowel disease to a pattern similar to that of normal mice, demonstrating an unexpected and excellent effect on microbiome health.

The composition of the present disclosure, when being a pharmaceutical composition, contains a pharmaceutically acceptable carrier. The pharmaceutically acceptable carrier is typically used at the time of formulation, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, and mineral oil.

The pharmaceutical composition of the present disclosure may further contain a lubricant, a wetting agent, a sweetening agent, a flavoring agent, an emulsifier, a suspending agent, a preservative, and the like, in addition to the above ingredients.

The pharmaceutical composition of the present disclosure may be used through parenteral administration, which may be, for example, intravenous administration, intraperitoneal administration, intramuscular administration, subcutaneous administration, or topical administration. Furthermore, oral administration, rectal administration, inhalation administration, intranasal administration, or the like may be possible. In a specific embodiment of the present disclosure, the pharmaceutical composition is for oral administration.

The appropriate dose of the pharmaceutical composition of the present disclosure varies depending on factors such as a formulating method, manner of administration, patient's age, body weight, gender, severity of disease, food, time of administration, route of administration, excretion rate, and response sensitivity, and an ordinarily skilled practitioner can easily judge and prescribe the dose effective for the desired treatment or prevention. According to a preferred embodiment of the present disclosure, the pharmaceutical composition is administered at a daily dose of 0.001-100 mg/kg.

The pharmaceutical composition of the present disclosure is formulated using a pharmaceutically acceptable carrier and/or excipient according to the method that is easily conducted by a person skilled in the art to which the present disclosure pertains, and the composition of the present disclosure may be prepared into a unit dosage form or may be inserted into a multi-dose container. Here, the dosage form may be a solution, a suspension, or an emulsion in an oily or aqueous medium, and may further include a dispersing agent or a stabilizer.

As used herein, the term “administration” means providing a given substance to a subject by any appropriate method. The administration route of the composition of the present disclosure can be oral or non-oral, as long as it can reach the target tissue by any general route described above. Additionally, the composition of the present disclosure may be administered using any device capable of delivering the active ingredient to target cells, tissues, or organs.

The term “subject”, as used herein, includes, but is not limited to, for example, humans, monkeys, cattle, horses, sheep, pigs, chickens, turkeys, quails, cats, dogs, mice, rats, rabbits, or guinea pigs, preferably mammals, and more preferably humans.

As used herein, the term “prevention” means any action that suppresses or delays the symptoms of an inflammatory disease by administering the composition according to the present disclosure.

As used herein, the term “treatment” means any action that alleviates or cures the symptoms of an inflammatory disease by administering the composition according to the present disclosure.

The pharmaceutical composition of the present disclosure includes a pharmaceutically effective amount of the particles of the present disclosure. The pharmaceutically effective amount means an amount sufficient to achieve the pharmacological effect of the particles mentioned above.

Additionally, the pharmaceutical composition of the present disclosure may further include active ingredients known in the art to have therapeutic effects on inflammatory bowel disease or chronic inflammatory diseases. Examples include steroids such as glucocorticosteroids, 5-aminosalicylic acid (5-ASA) drugs such as sulfasalazine and mesalazine, and anti-TNF-α monoclonal antibodies.

Another aspect of the present disclosure provides a food composition containing galacto-oligosaccharide composition as an active ingredient for the prevention or improvement of inflammatory bowel disease.

The food composition of the present disclosure may be prepared into forms of powder, granules, tablets, capsules, or beverages. For example, various foods like candy, drinks, gum, tea, vitamin complexes, or health supplements can be included.

The food composition of the present disclosure may include, in addition to the galacto-oligosaccharide composition as the active ingredient, ingredients commonly added during food preparation, such as proteins, carbohydrates, fats, nutrients, seasonings, and flavorings. Examples of carbohydrates include monosaccharides, such as glucose and fructose; disaccharides, such as maltose, sucrose, and oligosaccharides; and polysaccharides, such as dextrin and cyclodextrin, as well as conventional sugars and sugar alcohols like xylitol, sorbitol, and erythritol. Natural flavorings [such as thaumatin and stevia extract (e.g., rebaudioside A, glycyrrhizin)] and synthetic flavorings (e.g., saccharin, aspartame) can be used as flavorings. For instance, when being prepared as a drink, the food composition of the present disclosure may also include citric acid, liquid fructose, sugar, glucose, vinegar, malic acid, fruit juice, eucommia extract, jujube extract, and licorice extract in addition to the galacto-oligosaccharide composition.

A further aspect of the present disclosure provides an anti-inflammatory or inflammation-reducing feed composition containing the galacto-oligosaccharide composition.

In one embodiment of the present disclosure, the inflammation refers to intestinal inflammation.

The content regarding the galacto-oligosaccharide composition included in the food composition and feed composition of the present disclosure is the same as the content regarding the galacto-oligosaccharide composition included in the pharmaceutical composition. Therefore, the common content therebetween is omitted in this specification to avoid excessive complexity.

### Advantageous Effects of Invention

The galacto-oligosaccharide composition of the present disclosure demonstrates anti-inflammatory efficacy by improving DAI and reducing levels of inflammatory cytokines and myeloperoxidase (MPO) in a mouse model of inflammatory bowel disease. Therefore, the composition can be usefully employed as a drug or food product for the prevention, amelioration, or treatment of inflammatory bowel diseases.

### BRIEF DESCRIPTION OF DRAWINGS

FIG. 1 is a view illustrating the process of preparing the galacto-oligosaccharide composition of the present disclosure.

FIG. 2 is a high-performance size-exclusion chromatogram for analyzing the DP profile of the galacto-oligosaccharides of the present disclosure.

FIG. 3 is a spectrum for analyzing the DP of the galacto-oligosaccharides of the present disclosure.

FIG. 4 is a graph of the calculated DAI in mice with DSS-induced colitis tested in Example 3 of the present disclosure.

FIG. 5 is a graph of cecum lengths in mice with DSS-induced colitis tested in Example 3 of the present disclosure.

FIG. 6 is a graph of the levels of inflammatory cytokine IL-6 measured in the colon of the mouse model obtained in Example 3 of the present disclosure.

FIG. 7 is a graph of the levels of inflammatory cytokine TNF-α measured in the colon of the mouse model obtained in Example 3 of the present disclosure.

FIG. 8 is a graph of the levels of MPO, a marker of neutrophil infiltration, measured in the colon of the mouse model obtained in Example 3 of the present disclosure.

FIG. 9 presents results of staining intestinal tissues of mice with DSS-induced colitis using Alcian/PAS staining.

### BEST MODE FOR CARRYING OUT THE INVENTION

A better understanding of the present disclosure may be obtained through the following Examples, which are set forth to illustrate, but are not to be construed to limit the present disclosure.

### Examples

Unless otherwise stated, “%” used to indicate the concentration of a specific substance is (weight/weight) % for solid/solid, (weight/volume) % for solid/liquid, and (volume/volume) % for liquid/liquid throughout the specification.

### Example 1: Production of Galacto-Oligosaccharide

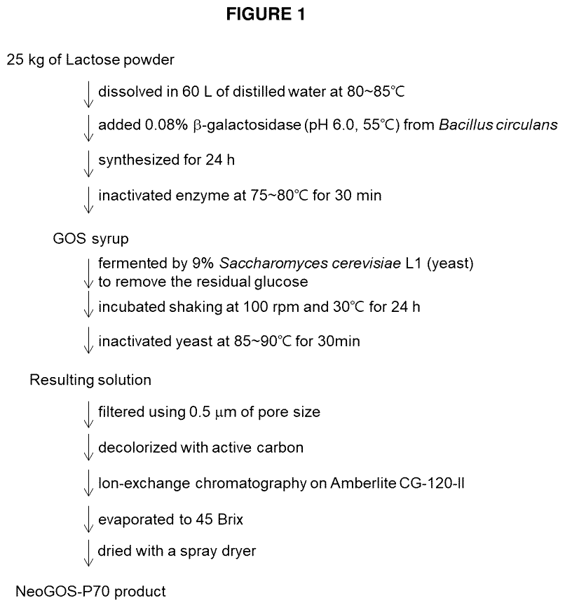

The galacto-oligosaccharides (GOS) used in the present disclosure were manufactured by Neocrema (Seoul, Korea). The manufacturing process is illustrated in FIG. 1.

As shown in FIG. 1, 25 kg of lactose powder was dissolved in purified water at 80-85° C. Then, 0.08% of β-galactosidase derived from Bacillus circulans was added to synthesize GOS over 24 hours. The enzyme was inactivated by heating at 75-80° C. for 30 minutes.

To remove any residual glucose left in the GOS syrup, 9% Saccharomyces cerevisiae L1 (yeast) was added to the syrup and shaken at 100 rpm at 30° C. for 24 hours. The yeast was inactivated by heating at 85-90° C. for 30 minutes.

The resulting solution was filtered using a 0.5 μm pore-size filter, decolorized with activated charcoal, and subjected to ion exchange chromatography using Amberlite CG-120-II. The solution was then concentrated until it reached a sugar content of 45 Brix and was spray-dried to produce the final galacto-oligosaccharide composition, named NeoGOS-P70. NeoGOS-P70 was used in subsequent Examples.

### Example 2: Composition Analysis of Galacto-Oligosaccharide Composition

### 2-1. Analysis of DP Profile (Degree of Polymerization Profile)

To analyze the DP profile of NeoGOS-P70, prepared in Example 1, a high-performance liquid chromatography (HPLC) system equipped with a refractive index detector (RID) was used. A Repromer Ca²⁺ column (Dr. Maisch GmbH, Ammerbuch, Germany) of the sulfonated styrene-divinylbenzene type was used at 85° C. Water (Millipore, Bedford, MA, USA) was used as the eluent at a flow rate of 0.5 mL/min. Since there is no commercially available standard product for DP analysis, standards such as galactose, glucose, maltose (Sigma Aldrich, St. Louis, MO, USA), and an oligosaccharide kit (Supelco, Bellefonte, PA, USA) were used to analyze the oligosaccharides above DP2. Lactose (disaccharide) was analyzed using an HPLC-RID system with the YMC-PACK polyamine II normal phase column (YMC Company, Ltd., Kyoto, Japan) at 40° C. The samples were eluted with a mixture of acetonitrile and 36% water at a flow rate of 1 mL/min. For comparison, the same analysis was performed on GOS compositions produced by Companies A and B.

The content of each DP (from DP2 to ≥DP4) was calculated using a standard curve based on maltose concentration. Additionally, the DP content excluding lactose was calculated by the formula: total DP content (%)-lactose content (%). DP content was calculated using data from the Repromer Ca²⁺ column, while lactose content was calculated using data from the YMC-PACK II column.

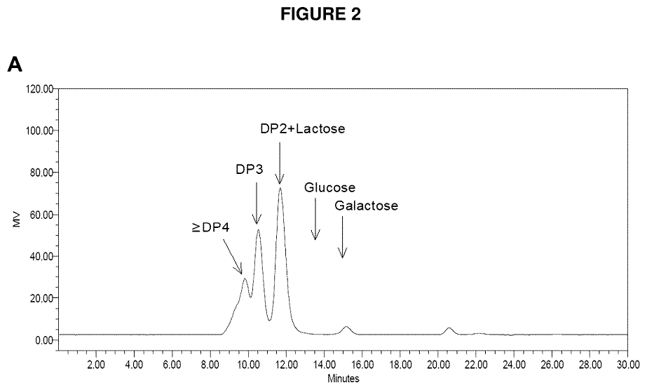

The results are presented in FIG. 2 and Table 1.

TABLE 1

| | | NeoGOS- | GOS from | GOS from |
|---|---|---|---|---|
| Sugar composition | | P70 | Company A | Company B |
| Mono-saccharide | Glucose | 0 | 2.5 | 2.3 |
| | Galactose | 1.7 | 1.5 | 1.6 |
| Disaccharides | Lactose | 20.9 | 19.1 | 18.5 |
| (DP2) | Other's | 22.8 | 22.6 | 23.5 |

| | | | |
|---|---|---|---|
| Trisaccharides (DP3) | 31.9 | 32.8 | 31.2 |
| Oligosaccharides (≥DP4) | 22.7 | 21.5 | 22.9 |
| Total GOS contents (%, DB) | 77.4 | 76.9 | 77.6 |
| Total Sugar contents (%, DB) | 100 | 100 | 100 |

DB: dry basis, (=% in solid content). As can be seen in FIG. 2 and Table 1, the galacto-oligosaccharide of the present disclosure contained about 22.8% of DP2 oligosaccharides (except for lactose), about 31.9% of DP3 oligosaccharides, and about 22.7% of DP4 or higher oligosaccharides, with the purity of galacto-oligosaccharide (except for lactose) amounting to 70% or higher.

### Example 2-2: MALDI-TOF/MS Analysis

To determine the molecular weight of NeoGOS-P70 prepared in Example 1, a matrix-assisted laser desorption ionization-time of flight mass spectrometry (MALDI-TOF/MS) in cation mode was used.

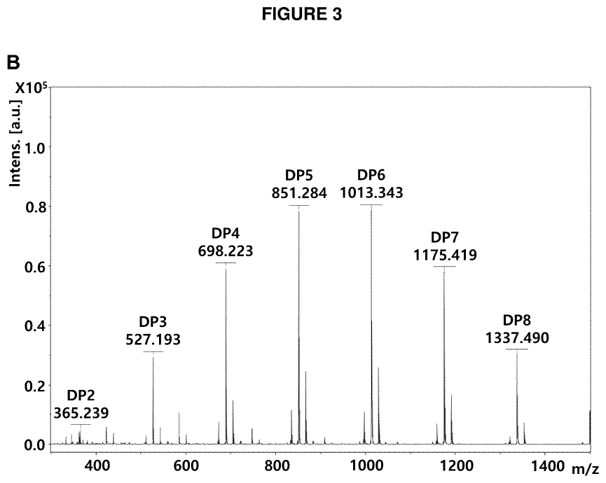

The galacto-oligosaccharides were first dissolved in purified water to a concentration of 10 mg/mL. Then, 1 μL of the sample was then mixed with 2,5-dihydroxybenzoic acid. After air-drying, the sample was analyzed using the Ultraflextreme MALDI-TOF/MS system (Bruker Daltonik, Bremen, Germany) with a mass spectrum range of 300-1500 Da. The results are shown in FIG. 3.

The mass spectrum of NeoGOS-P70, as presented in FIG. 3, revealed peaks at m/z 1337.490, 1175.419, 1013.343, 851.284, 689.223, 527.193, and 365.239, corresponding to the [M+Na]+ ions. These peaks align with octa-(DP8), hepta-(DP7), hexa-(DP6), penta-(DP5), tetra-(DP4), tri-(DP3), and di-saccharides (DP2), respectively.

These results indicate that NeoGOS-P70 prepared in Example 1 contains sugars ranging from DP2 to DP8.

### Example 2-3: Quantitative Analysis of 4′-Galactosyllactose

4′-Galactosyllactose (4′-GL) is a prominent component of human milk oligosaccharides (HMOs) and is included in the galacto-oligosaccharide (NeoGOS-P70) of the present disclosure.

To quantify the content of 4′-GL in the galacto-oligosaccharide (NeoGOS-P70) of the present disclosure, HPLC-ELSD (evaporative light scattering detector) equipped with an NH2P-50 4E column (Showa Denko K.K., Tokyo, Japan) was used. The elution was carried out with a solvent mixture of 75% acetonitrile and 25% water at a flow rate of 0.8 mL/min.

The results are presented in Table 2.

TABLE 2

| Sample | 4′-Galactosyllactose content (%) |
|---|---|
| NeoGOS-P70 | 16.50% |
| GOS from company A | 11.20% |
| GOS from company B | 10.40% |

Typically, 3′-, 4′-, and 6′-galactosyllactose are known as major components of human breast milk. In galacto-oligosaccharide (NeoGOS-P70) of the present disclosure, the retention time of 4′-galactosyllactose was 15.6 minutes, and its content was found to be (16.5±3.6) %. The DP analysis and quantitative analysis of 4′-galactosyllactose revealed that the galacto-oligosaccharides of the present disclosure have the composition presented in Table 2.

### Example 3: Effects of Galacto-Oligosaccharide Composition on Inflammatory Bowel Disease

The following experiments were conducted to verify the effects of the galacto-oligosaccharide composition of the present disclosure on inflammatory bowel disease.

### Example 3-1: Preparation of Experimental Animals with Inflammatory Bowel Disease

For use in this experiment, female BALB/c mice (7 weeks old) were purchased from Saeronbio Inc. (Gyeonggi-do, Korea) and acclimatized for 7 days under SPF conditions at Kyonggi University (temperature 23±2° C., humidity 50±5%, 12-hour light/dark cycle). All experiments adhered to the guidelines of the university's Animal Ethics Committee.

An acute colitis model was induced using dextran sodium sulfate (DSS).

Mice were divided into seven groups of seven mice each. 1) A normal control group received purified water orally for 28 days. 2) A colitis group received purified water for 28 days and 5% DSS solution freely from days 21 to 28 to induce colitis. The galacto-oligosaccharide of the present disclosure was administered at a dose of 3) 400 mg/kg and 4) 800 mg/kg to two groups of colitis-induced mice, respectively, for 28 days. In addition, to colitis-induced mice, the galacto-oligosaccharides from 5) company A and 6) company B were each administered at a dose of 800 mg/kg for 28 days while 7) the positive control group was administered 100 mg/kg of the anti-inflammatory agent 5-ASA (5-aminosalicylic acid) for 28 days. On the 29^{th} day, each mouse was sacrificed, and the length of the colon and small intestine and the weight of the spleen were measured and evaluated. After measurement, the colon tissue was cut into 1 cm lengths, and the cecum was excised and placed into separate Ep. tubes for storage in a deep freezer for future experiments. Portions of the remaining colon tissue were fixed in a 4% formalin solution.

### Example 3-2: Measurement of Disease Activity Index (DAI)

During the DSS treatment, colitis severity was determined using the disease activity index (DAI), which measures weight loss, stool consistency, rectal bleeding, and perianal blood presence according to the criteria in Table 3. After monitoring with the naked eye every two days for eight days, the scores were summed and calculated.

TABLE 3

| Score | Weight loss | Stool consistency | Occult/gross rectal bleeding |
|---|---|---|---|
| 0 | None | Normal | Normal |
| 1 | 1-5% | — | — |
| 2 | 5-10% | Loose stool | Hemoccult |
| 3 | 10-20% | — | — |
| 4 | 20% | Diarrhea | Gross bleeding |

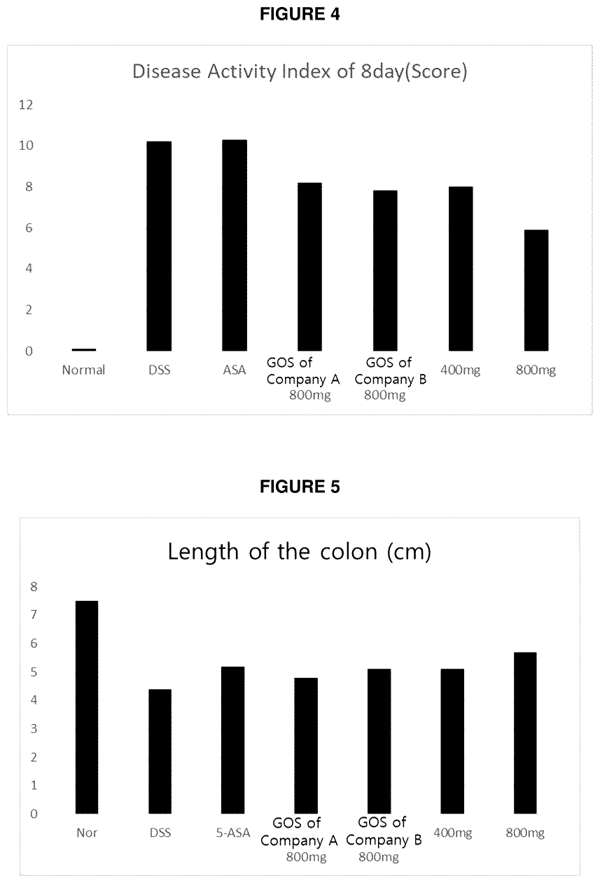

Results are summarized in Table 4 and depicted in FIG. 4.

TABLE 4

| Classification | Disease Activity Index of 8 day (Score) |
|---|---|
| Normal | 0.1 |
| DSS | 10.2 |
| 5-ASA | 10.3 |
| GOS 800 mg from company A | 8.2 |
| GOS 800 mg from company B | 7.8 |
| Neocrema GOS 400 mg | 8 |
| Neocrema GOS 800 mg | 5.9 |

Weight loss was observed in all acute colitis-induced groups except for the normal group. Additionally, the calculation of the Disease Activity Index (DAI) showed a significant increase in DAI in all groups except for the normal group, and the positive control group (5-ASA) did not show any colitis amelioration effect. However, the groups treated with galacto-oligosaccharides from Company A, Company B, and the present disclosure showed colitis amelioration effects. Notably, the group treated with 400 mg/kg of the galacto-oligosaccharide of the present disclosure showed a similar level of amelioration to the groups treated with 800 mg/kg of galacto-oligosaccharides from Company A and Company B. The group treated with 800 mg/kg of the galacto-oligosaccharide of the present disclosure showed the greatest amelioration effect.

Subsequently, on the 9^{th} day, the mice from each group were sacrificed to measure the length of the colon.

The results are shown in Table 5 and FIG. 5.

TABLE 5

| Classification | Colon length (cm) |
|---|---|
| Normal | 7.3 |
| DSS | 4.4 |
| 5-ASA | 5.2 |
| GOS 800 mg from company A | 4.8 |
| GOS 800 mg from company B | 5.0 |
| Neocrema GOS 400 mg | 5.1 |
| Neocrema GOS 800 mg | 5.7 |

As shown in Table 5 and FIG. 5, the colon length of all DSS-induced ulcerative colitis mice was shorter compared to the normal group. The positive control group (5-ASA) showed an efficacy in inhibiting the reduction of colon length. The groups treated with galacto-oligosaccharides from Company A and Company B, as well as the groups treated with 400 mg/kg and 800 mg/kg of the galacto-oligosaccharide of the present disclosure, also showed efficacy in inhibiting the reduction of colon length. Particularly, the group treated with 800 mg/kg of the galacto-oligosaccharide of the present disclosure showed the most outstanding efficacy in inhibiting the reduction of colon length, resulting in the longest measured colon length.

### Example 3-3: Measurement of Inflammatory Cytokines (IL-6, TNF-α) and MPO Levels in Colon Tissue

To measure the levels of inflammatory cytokines IL-6 (BD Biosciences, San Jose, CA, USA) and TNF-α (BD Biosciences), as well as myeloperoxidase (MPO) (Invitrogen, Carlsbad, CA, USA) in the colon tissues of DSS-induced colitis mice, the following experiments were conducted:

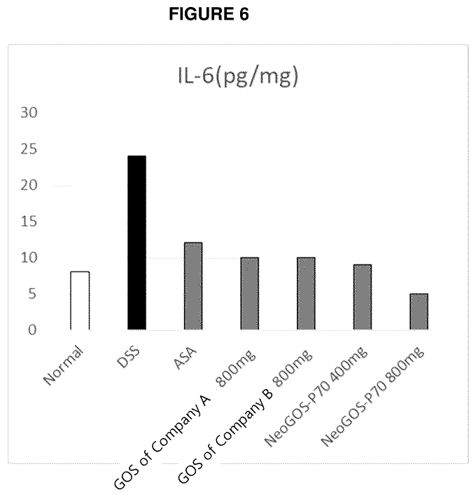

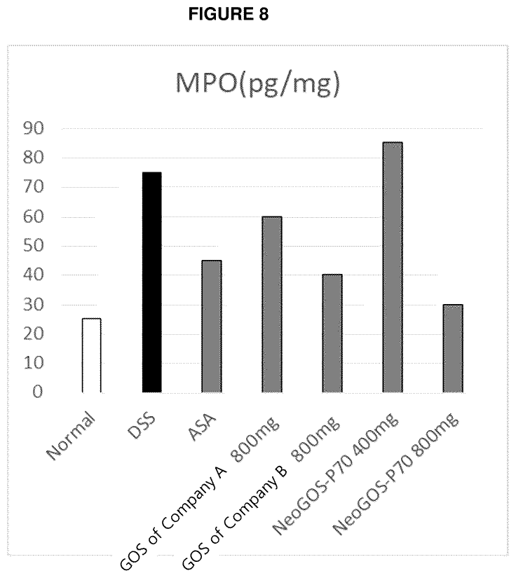

First, colon tissue samples were suspended in PBS containing a protease inhibitor (Roche Diagnostics Corp., Indianapolis, IN, USA) and homogenized uniformly using a TaKaRa BioMasher (Takara Bio Inc., Shiga, Japan). Then, the samples were centrifuged (1000×g, 20 min, 4° C.) and the supernatant was collected. Protein concentration was quantified using a bicinchoninic acid kit (Thermo Scientific, Waltham, MA, USA). The results are shown in Table 6 and FIGS. 6 to 8.

TABLE 6

| | IL-6 | TNF-α | MPO |
|---|---|---|---|
| Classification | (pg/mg) | (pg/mg) | (pg/mg) |
| Normal | 8 | 50 | 25 |
| DSS | 24 | 450 | 75 |
| 5-ASA | 12 | 100 | 45 |
| GOS 800 mg from company A | 10 | 70 | 60 |
| GOS 800 mg from company B | 10 | 75 | 40 |
| NeoGOS-P70 400 mg | 9 | 40 | 85 |
| NeoGOS-P70 800 mg | 5 | 50 | 30 |

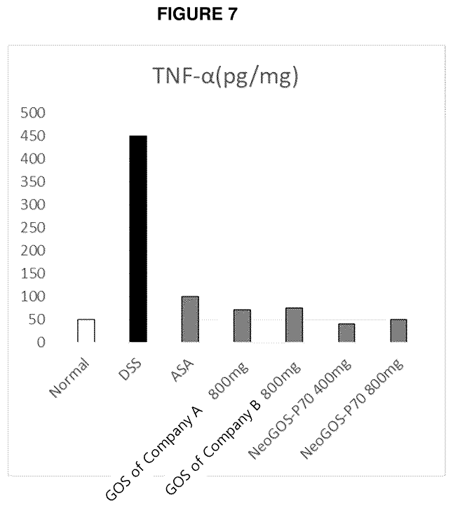

As shown in FIGS. 6 and 7, the expression levels of IL-6 and TNF-α were significantly elevated due to DSS administration. In contrast, in all experimental groups (5-ASA; Company A GOS 800 mg/kg, Company B GOS 800 mg/kg, the present invention's Neocrema GOS 400 mg/kg; the present invention's Neocrema GOS 800 mg/kg), the levels of IL-6 and TNF-α were greatly reduced. Notably, the group administered 800 mg/kg of the present invention's GOS showed the most dramatic decrease in the levels of IL-6 and TNF-α, which fell below the protein levels of the normal group.

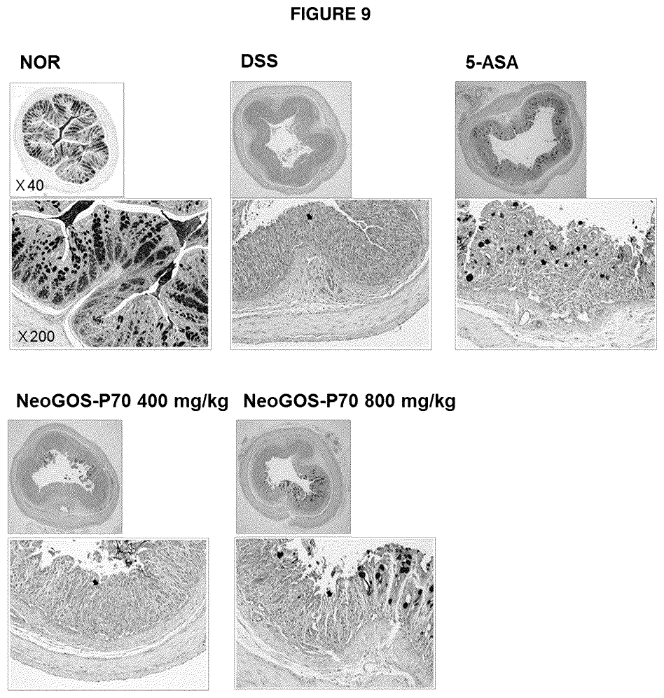

MPO is a peroxidase primarily found in neutrophils and is an indicator of neutrophil infiltration. As shown in Table 6 and FIG. 9, the group administered 800 mg/kg of the galacto-oligosaccharide of the present disclosure inhibited MPO to a level similar to that of the normal group.

Collectively, the galacto-oligosaccharide of the present disclosure was confirmed to have an anti-inflammatory effect in vivo by reducing the levels of pro-inflammatory cytokines (IL-6 and TNF-α) and decreasing MPO, a marker of neutrophil infiltration, in a DSS-induced colitis model.

### Example 3-4: Histological Analysis

A portion of the colon was fixed in 4% formalin solution for 24 hours. Paraffin-embedded tissue samples were cut into 5 μm sections and stained with Alician blue-periodic acid-Schiff (PAS) for histological examination. Observations were made under an optical microscope (Olympus BX53, Olympus, Tokyo, Japan) at 40× and 200× magnifications.

Mucin is the first line of host defense, distributed thickly on the intestinal surface, which is epithelial tissue. It protects the epithelial tissue of the intestinal surface from mechanical damage and chemical irritation, inhibits the invasion of pathogens and foreign substances, and facilitates their expulsion. Mucin exists in several subtypes based on its structure, with different tissue distributions depending on the subtype. The subtype most abundantly secreted in the colon is MUC2, which can be easily stained using alcian blue solution. Many previously reported studies have indicated that inducing colitis in mice by administering DSS in drinking water results in a rapid decrease in the mucus layer of the colon. Therefore, this study aimed to confirm the effect of administering the galacto-oligosaccharide of the present disclosure on mucin secretion in mice with DSS-induced colitis.

As shown in FIG. 9, the amount of mucin (blue) significantly decreased in DSS-treated mice compared to the normal group, and the loss of goblet cells (arrow) was observed. In contrast, in the groups administered the anti-inflammatory agent 5-ASA and the galacto-oligosaccharide of the present disclosure, the amount of mucin recovered, and goblet cells were observed compared to the DSS-treated group. These results confirm that the administration of the galacto-oligosaccharide of present disclosure recovers the reduced mucin in the DSS-induced colitis model and helps maintain the function of the intestinal barrier.

### Example 3-5. Evaluation of Short Chain Fatty Acid (SCFA) Production Efficiency in Intestine

To determine the effect of administering the galacto-oligosaccharide of the present disclosure on the intestinal short chain fatty acids in DSS-induced mice, 5-ASA and the GOS of the present disclosure were administered at low (7 mg/mouse), medium (14 mg/mouse), and high (21 mg/mouse) concentrations to DSS-induced mice according to the schedule described in Example 3-1. Afterward, the cecums of the mice were collected, homogenized with a homogenizer, and suspended in 80% methanol. The samples were then centrifuged at 13,000 rpm for 10 minutes, diluted to a final concentration of 1 g/mL, and filtered through a 0.45 μm syringe filter.

Quantitative analysis of SCFAs was performed by preparing each STD at different concentrations and using 2-ethylbutyric acid as the internal standard (ISTD). The standard curves were calculated based on the peak area ratios. In brief, the SCFA content was measured using gas chromatography (GC) equipped with a DB-FFAP capillary column to determine the production capacity of six SCFAs (acetic acid, propionic acid, butyric acid, valeric acid, caproic acid, and heptanoic acid). The GC analysis conditions were as follows: 100° C. (0.5 min), 100° C.→180° C. (8° C./min), 180° C. (1.0 min), 180° C.→200° C. (20° C./min), 200° C. (5.0 min).

The results are shown in Table 7.

As indicated in Table 7, the total SCFA content in the NC (Negative Control) treated with 5% DSS alone decreased by approximately 54.9% compared to the normal control NT, with the butyric acid content showing a significant decrease by approximately 12-fold among the six SCFAs. In the PC (Positive Control) group, all SCFAs except acetic acid showed a slight increase compared to the NC treated with 5% DSS alone. On the other hand, the low concentration of GOS (7 mg/mouse) and the medium concentration of GOS (14 mg/mouse) demonstrated a dose-dependent increase in total SCFA content compared to the NC treated with 5% DSS alone, with a significant increase in the content of butyric acid, which has been previously reported to have various physiological functions. However, the high concentration of GOS (21 mg/mouse) showed a tendency to decrease significantly compared to the NC. Therefore, it was ultimately confirmed that the medium concentration of GOS (14 mg/mouse) had the best SCFA production efficiency.

TABLE 7

| | | Short Chain fatty acid production in cecum (μM) | | | | | | |
|---|---|---|---|---|---|---|---|---|
| | Dose | | Propionic | | Valeric | Caproic | Heptanoic | |
| Samples | (mg/kg) | Acetic acid | acid | Butyric acid | acid | acid | acid | Total SCFA |
| NT | — | 18300 (62.7) | 796 (2.7) | 10086 (34.6) | — | — | — | 29182 (100) |
| NC | — | 11301 (85.9) | 1012 (7.7) | 841 (6.4) | — | — | — | 13154 (100) |
| PC | 100 | 8917 (73.3) | 1321 (10.9) | 1923 (15.8) | — | — | — | 12161 (100) |
| GOS | 7 | 9445 (74.1) | 1212 (9.0) | 2267 (16.9) | — | — | — | 13424 (100) |
| | 14 | 10586 (75.5) | 1197 (8.5) | 2245 (16.0) | — | — | — | 14028 (100) |
| | 21 | 6290 (78.9) | 637 (7.9) | 1043 (13.1) | — | — | — | 7970 (100) |

What is claimed is:

1. A pharmaceutical composition comprising a galacto-oligosaccharide composition as an active ingredient for prevention or treatment of inflammatory bowel disease.

2. The pharmaceutical composition of claim 1, wherein the galacto-oligosaccharide composition comprises 4′-galactosyllactose in an amount of 12% to 25% by weight, based on the total weight thereof.

3. The pharmaceutical composition of claim 1, wherein the galacto-oligosaccharide composition has a purity of 70% to 95%.

4. The pharmaceutical composition of claim 1, wherein the galacto-oligosaccharide composition comprises monosaccharides in an amount of 5% by weight or less, based on the total weight thereof.

5. The pharmaceutical composition of claim 1, wherein the inflammatory bowel disease is selected from the group consisting of ulcerative colitis, Crohn's disease, intestinal Behcet's disease, indeterminate colitis, bacterial enteritis, viral enteritis, amoebic colitis, hemorrhagic rectal ulcers, leaky gut syndrome, ischemic colitis, and tuberculous enteritis.

6. The pharmaceutical composition of claim 1, wherein the pharmaceutical composition promotes production of short chain fatty acids in a gut.

7. A food composition comprising a galacto-oligosaccharide composition as an active ingredient for prevention or amelioration of inflammatory bowel disease.

8. An anti-inflammatory or inflammation-reducing feed composition comprising a galacto-oligosaccharide composition.
